(19) United States
(12) Patent Application Publication
(10) Pub. No.: US 2004/0115090 A1
(43) Pub. Date: Jun. 17, 2004

(54) Sanitized organic waste

(76) Inventors: Hans Andersson, Norrkoping (SE); Tomas Kjellquist, Linkoping (SE); Conny Hellgren, Norrkoping (SE); Robert Taflin, Uppsala (SE)

Correspondence Address: SWIDLER BERLIN SHEREFF FRIEDMAN, LLP, 3000 K STREET, NW, BOX IP, WASHINGTON, DC, 20007, US

(21) Appl. No.: 10/679,315

(22) Filed: Oct. 7, 2003

Related U.S. Application Data
(60) Provisional application No. 60/416,584, filed on Oct. 8, 2002.

(51) Int. Cl. A01N 1/00; A61L 2/00

(52) U.S. Cl. 422/1; 422/5; 422/38; 422/40

(57) ABSTRACT

Methods for sanitizing organic waste without introducing exogenous heat or chemicals are provided, which involve mixing organic waste with nutrient rich organic material to form a porous matrix that can be maintained at a temperature and duration sufficient to reduce the amount of living microorganisms in the mixture to sanitary levels. The invention also includes dewatering the organic waste prior to the sanitizing process, using various organic fibrous materials.

### FIELD OF THE INVENION

The present invention relates to methods for sanitizing organic waste as well as the sanitized organic waste produced by such methods. These methods involve mixing organic waste with nutrient rich organic material to form a porous matrix that can be maintained at a temperature and duration sufficient to reduce the amount of living microorganisms in the mixture to sanitary levels without introducing exogenous heat or chemicals. The invention also includes dewatering the organic waste prior to the sanitizing process, using various organic fibrous materials.

### BACKGROUND OF THE INVENTION

Untreated wastewater and sludge can contain harmful or unwanted microorganisms, including various pathogens. This can result in foul odor, nonhygienic conditions, or other undesirable characteristics. One goal in wastewater and sludge treatment is to obtain a product in which the level of harmful or unwanted microorganisms is significantly reduced.

Various methods exist for treating wastewater or sludge, but such methods typically depend upon introducing exogenous heat or chemicals. See, e.g., U.S. Pat. No. 5,853,450, U.S. Pat. No. 4,902,431, and U.S. Pat. No. 4,781,842. Other a treatment methods, for example, involve adding pressure and heat, followed by dehydration. See U.S. Pat. No. 6,197,081. Yet another method for treating wastewater or sludge, for example, involves adding heat through a continuous flow pasteurization system. See U.S. Pat. No. 6,103,191. Other methods also involve the pretreatment step of dewatering wastewater or sludge by adding various chemicals, such as polymers.

None of these methods, however, provides for the cost-efficient treatment of organic waste in a manner sufficient to reduce the amount of living microorganisms in the mixture to sanitary levels without introducing exogenous heat or chemicals. A valuable contribution to the art, therefore, would be methods for sanitizing organic waste without introducing exogenous heat or chemicals.

### SUMMARY OF THE INVENTION

Accordingly, an objective of the present invention involves providing methods for sanitizing organic waste without introducing exogenous heat or chemicals. The present invention accomplishes this and other objectives, for example, by providing methods comprising mixing organic waste with nutrient rich organic material to form a porous matrix that can be maintained at a temperature and duration sufficient to reduce the amount of living microorganisms in the mixture to sanitary levels without introducing exogenous heat or chemicals. The invention also includes dewatering the organic waste prior to the sanitizing process, using various organic fibrous materials.

### BRIEF DESCRIPTION OF THE DRAWINGS

The figures and accompanying descriptions depict various embodiments of the present invention.

FIG. 1 illustrates two embodiments of the present invention, one involving an uncovered matrix (an open process), and the other involving a covered matrix (a closed process).

FIG. 2 illustrates further embodiments of the present invention that can include dewatering the organic waste with organic fibrous material, which can also fulfill the purpose of nutrient rich material in the porous matrix.

FIG. 3 illustrates an embodiment of the present invention with a porous matrix.

FIG. 4 provides an example of temperature maintenance in a porous matrix comprising a mixture of organic waste with nutrient rich organic material.

FIG. 5 provides an example of temperature development in a porous matrix in an embodiment of the present invention during mixing and aeration that can include using a special bucket with grinders compared to mixing with a traditional bucket.

FIG. 6 provides an example of temperature development in a porous matrix in an embodiment of the present invention that can include a closed sanitization process.

FIG. 7 provides an example of temperature development in a porous matrix in an embodiment of the present invention that can comprise a mixture of organic waste (50 vol-%) with nutrient rich material (50 vol-%).

FIG. 8 illustrates the amount of biodegradable organic material in percentage of the total amount of organic material that is degraded within 96 hours in liquid from ruminants in different nutrient rich organic materials.

FIG. 9 provides an example of temperature development in porous matrixes in embodiments of the present invention that can be covered with isolation material comprising of sewage sludge mixed with 50 vol-% bark and 75 vol-% bark, respectively.

FIG. 10 provides an example of a typical temperature distribution within a porous matrix of the present invention at the stages of mixing and aeration.

FIG. 11 further illustrates examples of temperature distribution in porous matrixes of the present invention that can include a young and an old sanitization matrix.

FIG. 12 further illustrates one embodiment of the present invention that can include mixing organic waste with organic fibrous material sufficient to promote insignificant odor and high dry matter content, or without introducing significant amounts of exogenous chemicals, and dewatering said mixture, and the organic fibrous material in said mixture may also be further used as nutrient rich material facilitating the sanitizing process.

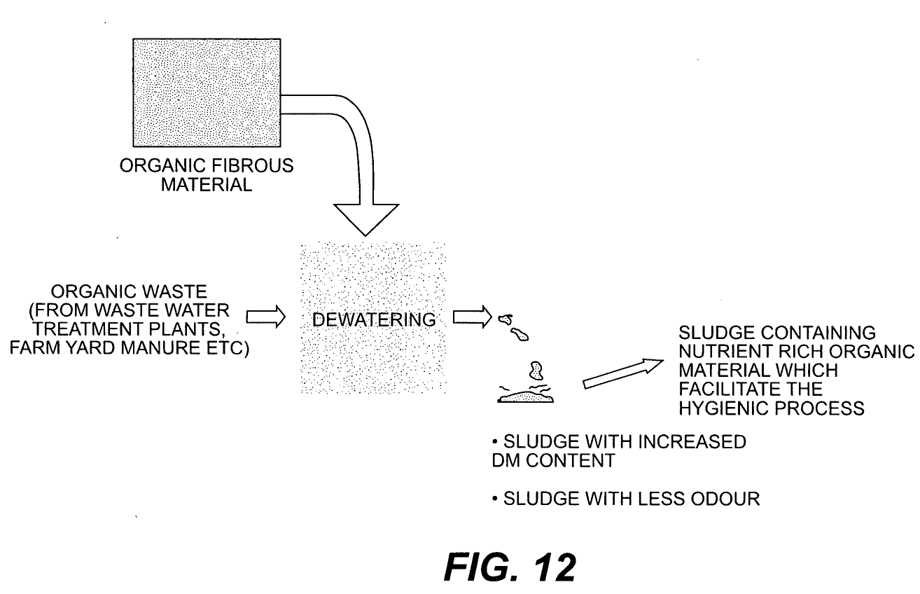

### DETAILED DESCRIPTION OF THE INVENTION

In one embodiment, the present invention involves a method for sanitizing organic waste comprising mixing organic waste with nutrient rich organic material, forming a porous matrix comprising the mixture of organic waste and nutrient rich organic material, maintaining the matrix, without introducing exogenous heat or chemicals, at a temperature and duration sufficient to reduce the amount of living microorganisms in the mixture to sanitary levels, and recovering a sanitized mixture.

In another embodiment, the present invention involves a method for sanitizing organic waste comprising mixing organic waste with nutrient rich organic material, forming a porous matrix comprising the mixture of organic waste and nutrient rich organic material, covering the matrix, maintaining the matrix, without introducing exogenous heat or chemicals, at a temperature and duration sufficient to reduce the amount of living microorganisms in the mixture to sanitary levels, and recovering a sanitized mixture.

In one other embodiment, the present invention involves a method for sanitizing organic waste comprising mixing organic waste with nutrient rich organic material, forming a porous matrix comprising the mixture of organic waste and nutrient rich organic material, maintaining the matrix, without introducing exogenous heat or chemicals, at a temperature and duration sufficient to reduce the amount of living microorganisms in the mixture to sanitary levels, homogenizing the mixture, and recovering a sanitized mixture.

In yet another embodiment, the present invention involves a method for sanitizing organic waste comprising mixing organic waste with nutrient rich organic material, dewatering the mixture, forming a porous matrix comprising the mixture of organic waste and nutrient rich organic material, maintaining the matrix, without introducing exogenous heat or chemicals, at a temperature and duration sufficient to reduce the amount of living microorganisms in the mixture to sanitary levels, homogenizing the mixture, and recovering a sanitized mixture.

In an embodiment, the present invention involves a method for sanitizing organic waste comprising mixing organic waste with nutrient rich organic material, dewatering the mixture without introducing significant amounts of exogenous chemicals, forming a porous matrix comprising the mixture of organic waste and nutrient rich organic material, maintaining the matrix, without introducing exogenous heat or chemicals, at a temperature and duration sufficient to reduce the amount of living microorganisms in the mixture to sanitary levels, homogenizing the mixture, and recovering a sanitized mixture.

In another embodiment, the present invention involves sanitized organic waste obtained by a method comprising mixing organic waste with nutrient rich organic material, forming a porous matrix comprising the mixture of organic waste and nutrient rich organic material, maintaining the matrix, without introducing exogenous heat or chemicals, at a temperature and duration sufficient to reduce the amount of living microorganisms in the mixture to sanitary levels, and recovering a sanitized mixture.

In one other embodiment, the present invention involves a method of dewatering organic waste comprising mixing organic waste with organic fibrous material sufficient to promote insignificant odor and high dry matter content, or without introducing significant amounts of exogenous chemicals, and dewatering said mixture.

As used herein, the following terms may be ascribed particular meanings, which might or might not be consistent with the meaning otherwise common to one skilled in the art:

“Sanitizing” may refer to the property by which a treatment method renders a product that exhibits a significant reduction in the levels of harmful or unwanted microorganisms originally present in the starting material. For example, “sanitized organic waste” may refer to treated organic waste, which contains no viable Salmonella spp in 50 g (wet weight) and exhibits at least a 6 Log₁₀ reduction in Escherichia coli from that in untreated organic waste. See Working Document on Sludge, 3^{rd} draft, EC 2000, ENV.E.3/LM, Annex I. Alternatively, for example, “sanitary level” may refer to the level of Escherichia coli in treated organic waste of less than 5×10² CFU/g. See id.

“Organic waste” may refer to, for example, (i) sludge, (ii) organic sludge, (iii) bio-organic sludge, (iv) organic manure, and (v) contaminated soil. “Sludge” may refer to, for example, (i) residual sludge from sewage plants treating domestic or urban wastewater as well as from other sewage plants treating wastewater of a composition similar to domestic and urban wastewater; (ii) residual sludge from septic tanks and other similar installations for the treatment of sewage; and (iii) residual sludge from other sewage plants or sources. “Organic sludge” may refer to, for example, sludge derived from industrial products and byproducts comprising organic material, for example sludge from food-processing industry, or otherwise biodegradable materials, not necessarily of biological origin. “Bioorganic sludge” may refer to, for example, an organic sludge composed of materials such as sludges resulting from production of antimicrobials and other pharmaceutical products; bacterial fermentation sludges; sludges resulting from production of beer and wine; mushroom compost waste; paper mill sludges; sludges that contain microorganisms resulting from recycled organic products such as paper products; sludges resulting from the growth of microorganisms for the production of chemicals and organics; industrial sludges and byproducts resulting from the production of microbial products and foodstuffs; sludges resulting from the animal slaughter industry, preferably those that are digested or otherwise broken down by microorganisms; and sludges containing animal manures, such as chicken, pig, cattle, or horse manure.

“Treated organic waste” may refer to organic waste that has undergone biological, chemical or heat treatment, long-term storage, or any other method that renders the organic waste with a significantly reduced fermentability or decrease in the associated health hazards. The sanitization may also be referred to as “hygienization,” one example of which can be a 6 Log₁₀ reduction of a microorganism, such as Salmonella senftenberg W 775. See Working Document on Sludge, 3^{rd} draft, EC 2000, ENV.E.3/LM, Annex I. “Nutrient rich” may refer to a high proportion of biodegradable carbon sources, which can facilitate microbial conversion under various oxygen tension states, such as aerobic, facultative aerobic, facultative anaerobic, or anaerobic conditions.

“Organic fibrous material” may refer to carbon and fibrous rich material, which can facilitate the dewatering process by binding to the organic and inorganic material in the slurry. Organic fibrous material can also be nutrient rich material with a high proportion of fibrous and desired dewatering characteristics.

“Porous” may refer to a state of sufficient aeration, including oxygenation, to facilitate recovery of sanitized organic waste according to the present invention. For example, the inherent composition of materials can render a porous structure or ventilation shafts or pockets can be provided to achieve a porous structure.

“Matrix” may refer to a structure of any size, form or composition suitable to incorporate a mixture of organic waste and nutrient rich organic material. One example of a matrix may be a “hygienic madras,” which may refer to an embodiment of the present invention in which a mixture of organic waste and nutrient rich organic material is contained in a matrix of discrete size, volume and proportional dimension.

“Agriculture” may refer to the growing of all types of crops (commercial food crops as well as others), including those for stock-rearing purposes.

“Use of organic waste” may refer to the application of organic waste material, including the topical or integral introduction of organic waste with soil.

The present invention is not limited to the particular methodologies, compounds, materials, manufacturing techniques, uses, and applications described herein, as these can vary in ways understood by those skilled in the art. The terminology used herein is used for the purpose of describing particular embodiments only, and is not intended to limit the scope of the present invention. As used herein (as well as in the claims), the singular forms “a,” “an,” and “the” include the plural reference unless the context clearly indicates otherwise. In addition, all cited references cited are incorporated by reference in their entirety.

The present invention can provide for the efficient and/or cost-effective sanitization of organic waste without introducing exogenous heat or chemicals by relying in whole or part upon the microbial content of the organic waste or nutrient rich organic material, and the actions or transformation activities of such microorganisms. Microbial transformation and heat generation can facilitate the significant reduction in harmful or unwanted microorganisms in the organic waste to achieve a sanitized product. For example, the mixing of organic waste with nutrient rich organic material in appropriate relative amounts can create suitable conditions to support rapid microbial growth, which, without introducing exogenous heat or chemicals, in a suitable porous matrix can generate heat sufficient over proper time to sanitize the organic waste to an acceptable sanitary or hygienic level. In certain embodiments, the microorganisms that can contribute to the heat generation may originate or derive from those found in the component material or may be added to the mixture.

The microorganisms of the present invention can include, for example, Actinomycetes such as Actinobifida chromogena, Microbispora bispora, Micropolyspora faeni, Nocordia sp, Pseudocardia thermophilia, Streptomyces rectus, S. thermofuscus, S. thermoviolaceus, S. thermovulgaris, S. violaceus-ruber, Thermoactinomyces sacchari, T. vulgaris, Thermomonospora curvata, and T. viridis. The microorganisms of the present invention can also include, for example, fungi such as Aspergillus fumigatus, Humicola grisea, H. insolens, H. lanuginosa, Malbranchea pulchella, Myriococcum themophilum, Paecilomnyces variotti, Paputaspora thermophila, Scytalidium thermophilim, and Sporotrichum thermophile. Further, the microorganisms of the present invention can include, for example, bacteria such as Alcaligenes faecalis, Bacillus brevis, B. circulans complex, B. coagulans type A, B. coagulans type B, B. licheniformis, B. megaterium, B. pumilus, B. sphaericus, B. stearothermophilus, B. subtilis, Clostridium thermocellum, Escherichia coli, Flavobacterium sp, Pseudomonas sp, Serratia sp, and Thermus sp.

The following common pathogens and parasites can, for example, be killed during the sanitizing process of the present invention: Salmonella typhosa, Trichinella spiralis (larvae), Brucella abortus or B. suis, Streptococcus pyogenes, Shigella sp, Micrococcus pyogenes, Mycobacterium tuberculosis, Corynebacterium diptheriae, Entamoeba histolytica (cysts), Taenia saginata, Necator americanus, and Ascaris lumbricoides (eggs).

The present invention can provide a greater and more stable microbial transformation and heat generation associated with organic waste treatment than previously known methods. As a consequence, the present invention can sanitize organic waste without introducing exogenous heat or chemicals. The heat that can be generated by the methods of the present invention can significantly reduce the amount of harmful or unwanted microorganisms in organic waste. Further, the embodiments of the present invention that involve dewatering the organic waste can create a preferred homogenous blend of the component materials, which can facilitate a faster and more even microbial transformation and heat generation to treat the organic waste.

Various examples of organic waste that may be sanitized according to the present invention include sludges; contaminated soil (e.g., contaminated by oil or other matter such as grocery-industry products or byproducts); contaminated animal-feed; cadavers and butcher waste; cattle feedlot and swine manure that might contain feces, urine, wastewater, and animal feed; and composted manure used in organic farming practices.

The methods of sanitizing organic waste according to the present invention may be optimized by varying the mixture of organic waste and nutrient rich organic material. For example, a mixture by volume of about 30% to 80% nutrient rich organic material with organic waste can be suitable for microbial action as well as aeration. The nutrient rich organic material thus provides sufficient nutrients, for example, biodegradable carbon, for the microorganisms to grow and generate heat. Contrary to what was believed in the prior art, the nutrient content of organic waste, for example, sewage sludge, is not very high. By adding nutrient rich organic material to the matrix, the methods of the present invention therefore overcome this deficiency. In addition, the nutrient rich organic material can improve achieving and maintaining a structure of the mixed materials that contains and withholds suitable aeration for the microbial action. Additional porosity of the matrix to help achieve suitable aeration or oxygenation may be accomplished by adding structural material such as bark, or by mechanical mixing or other agitation of the mixture. The present invention provides methods for sanitizing organic waste without the introduction of exogenous heat or chemicals. The organic waste can comprise solid and liquid components. In various embodiments of the present invention, the dry matter content of the organic waste in weight percentage can be, for example, about 10-90%, about 20-60%, about 30-50%, or about 40%. Examples of organic waste that can be sanitized include sewage sludge, bioorganic sludge, septic tank sludge, industrial sludge, domestic refuse, products from food-industry, e.g., slaughter-house waste, products from retail-stores, products from restaurants, products from fodder-industry, e.g., fish-mill, meat-mill etc., animal or farm manure, and contaminated soil.

The nutrient rich organic material can comprise, for example, plant residues, products from forest and paper industry such as bark, debarking reject, knot reject, fiber sludge (primary sludge), bio sludge, chemical sludge, mixed sludge, debarking reject, screen reject, recycled fiber such as, for example, dip sludge, and screenings, and wood chops. Further, the nutrient rich organic material can comprise, for example, products from saw-mill industry, such as saw dust and planning shavings etc., and products from agriculture such as hay, straw, and other crop residues from, for example, grain, sugar cane, maize, and rice. Products from other food-processing industry such as, for example, whey, peel, coco fiber, etc., and products from vegetables and fruit processing industry such as, for example, rice reject and olive seeds, can also be used as the nutrient rich organic material. Other examples of useful nutrient rich organic material are material such as garden and tree waste, and peat, etc.

In certain embodiments, the nutrient rich organic material can include adjusting components that optimize the conditions for the microbial action, such as pH, nutrients, water, and microbes. Adjusting components can include, for example, chalk, limestone, lime sludge, lime mud, burnt lime mud, green liquor sludge, ashes, mineral fertilizer, and material from fresh or sea water that is rich in nutrients (e.g., algae and organic manure), or solutions, suspension or other mixtures of these components. The method of the present invention can also include inoculating microorganisms to the matrix for enhancing the sanitizing process and the generation of heat.

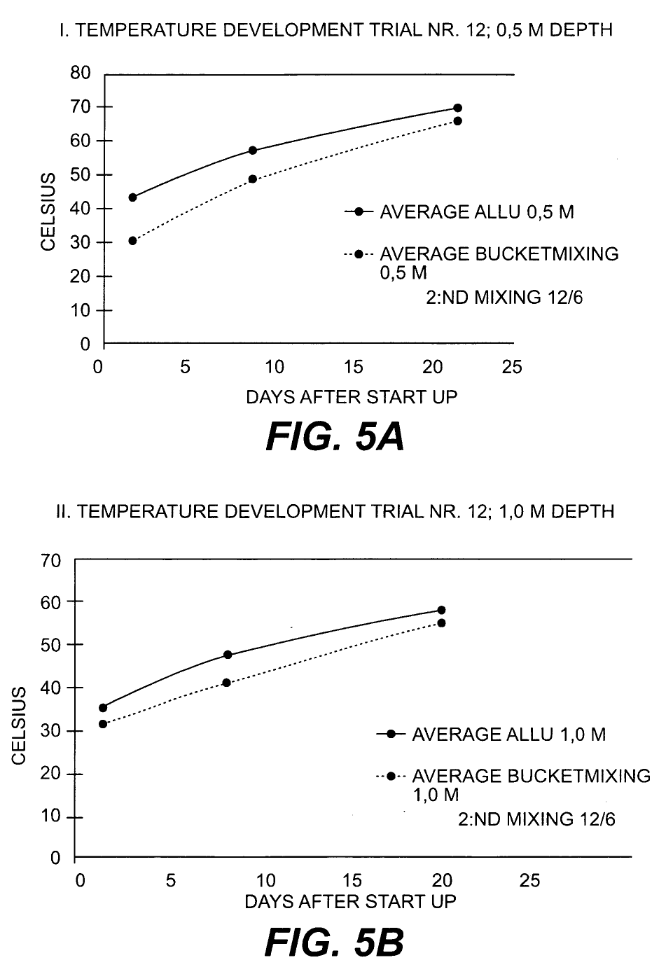

The porous matrix can be formed from the mixture of organic waste with nutrient rich organic material. The matrix thus can be sufficiently aerated to optimize the conditions for the microorganisms in the matrix to grow and generate heat. In forming the porous matrix, the aeration process can, for example, take place while mixing the organic waste with the nutrient rich organic material and/or further while the mixture is formed into a porous matrix and/or while in the porous matrix. Formation of the porous matrix, or its aeration, can be accomplished by physical manipulation using different techniques, for example, using a digger, loader, shoveling equipment, or a composting machine that can lift or turn the material over. The use of a traditional bucket on the loader and using specially designed buckets with a mixing unit, such as grinders, are examples of such aeration techniques, whose temperature developing effect can be seen from FIG. 5. Insignificant for the end result, the immediate aeration effect may vary depending on the technique and machinery used. A digger, for example, may be slightly more efficient compared to a loader because it mixes the material twice whereas a loader mixes the material once. In addition, shoveling equipment with rotating axles within the shovel can be used to mix and aerate the material before it falls through the shovel, which can occur at a predetermined fractionation size (between 5 mm and 200 mm, for example). Furthermore, air can be supplied to the porous matrix by ventilating the matrix, for example, by installing pipes or hoses. Suitable aeration at the mixing stage of the sanitization process, that is, the amount of aeration and its distribution within the mixture, without substantially interfering with the structure, can enhance the sanitization process because it may facilitate the generation of heat.

The matrix may be suitably “porous” when it facilitates a sufficient amount of aeration to support microbial growth and heat generation to accomplish the sanitization. Among the factors to adjust to consider can be the carbon/nitrogen ratio, structure, and volume weight of the mixture of organic waste and nutrient rich organic material. In a preferred embodiment, the matrix can be created with materials that include bark and fiber sludge. Dry and fibrous materials, such as sawdust, leaves, finely chopped straw, or peat moss, also provides for structural support that in combination with active mixing can make the matrix porous. The porous matrix, for example, can comprise a hygienic madras. The volume of the hygienic madras can exceed 5 m³. Various examples of the madras size can be 100 m³, 500 m³, 1000 m³, and 10000 m³.

The appropriate duration to accomplish sanitizing the organic waste according to the present invention can take into account the prevailing climatic conditions in the area where the treatment is performed. In open process embodiments, for example, the effects of the surrounding environment, such as, for example, weather and wind, can affect the temperature of the matrix. Generally, the temperature rise in the matrix starts within a few days to a couple of weeks depending on the structure and origin of integrated material. The temperature will rise in time with the microbial transformation of the material. At the peak of transformation the temperature might be, for example, in the range of 55-80° C. The temperature might, however, decrease gradually for the outermost 25 cm area surrounding the matrix in an open process. The temperature in that area can be below the lowest of the above-exemplified temperature intervals, e.g., below 60° C. This material can make up about 5-10% of the total material in the matrix.

In a preferred embodiment, the method can generate a temperature equal to, or greater than, 55° C. The duration of the sanitization can be between 12 hours and 1 year. The appropriateness of the conditions and duration can be assessed by the observation of a 6 Log₁₀ reduction of a test organism such as Salmonella senftenberg W 775 in the recovered organic waste as compared to that in the untreated organic waste.

Various examples of temperature and duration parameters can include maintaining the matrix, for example, at 70° C. for 1 day (24 hours), at 65° C. for 3 days (72 hours), at 60° C. 6 days (144 hours), or at 55° for 14 days (336 hours). Higher and lower temperatures can be acceptable with corresponding durations such that the treated organic waste, for example, contains no viable Salmonella spp in 50 g (wet weight), or has achieved at least a 6 Log₁₀ reduction in Escherichia coli to less than 5×10² CFU/g. Various parameters for rapid composting can be instructive. See On Farm Composting, Rynk et al., 1992, Northeast Regional Agricultural Engineering Service, Ithaca, N.Y. (1992).

In various embodiments, the present invention may involve an open process or a closed process, where the optional use of a cover, which can be placed over, around, or encapsulating the matrix, represents the closed process. The cover can be a single layer or constitute multiple layers, and be composed of various materials. A layer covering the matrix of a closed process can be made up of, for example, the following: a layer of the component organic materials of the matrix either by it self or in diverse mixtures; a layer by construction; a layer of inorganic compounds such as, for example, steel, rubber, plastic, fabrics, stones, etc.; a layer of organic compounds that can also be used as the nutrient rich organic material within the mixture, as well as a layer of other organic compounds such as, for example, color room reject, planks, fabrics, etc.; and a layer of soil.

The cover preferably may be two layers consisting of an outer layer that protects the matrix from water, snow, and ice, and an inner layer having appropriate isolating capabilities. The outer layer can thus consist of organic or inorganic material and also, for example, be a building or similar construction. In any event, the cover should embody suitable characteristics to facilitate the maintenance of suitable temperatures within the matrix to accomplish the sanitization according to the present invention. Regarding certain materials, one layer may achieve the isolation and protection. For example, the cover can be between 0.3 and 2 meters in thickness and contain materials such as fiber sludge, bark, or the like. The cover may preferably be about 0.5 meters to isolate a matrix during a closed process, in particular during colder climate conditions, to guarantee that all the component material to be sanitized achieves the desired temperature, also in the outermost layer in the matrix. The cover can also prevent contamination of the organic waste by the external environment during the sanitization method. The external environment in this regard can include microorganisms, insects, birds, animals, debris, weather-related matter, etc. The cover can thus, for example, prevent vectors to infect or re-infect the material.

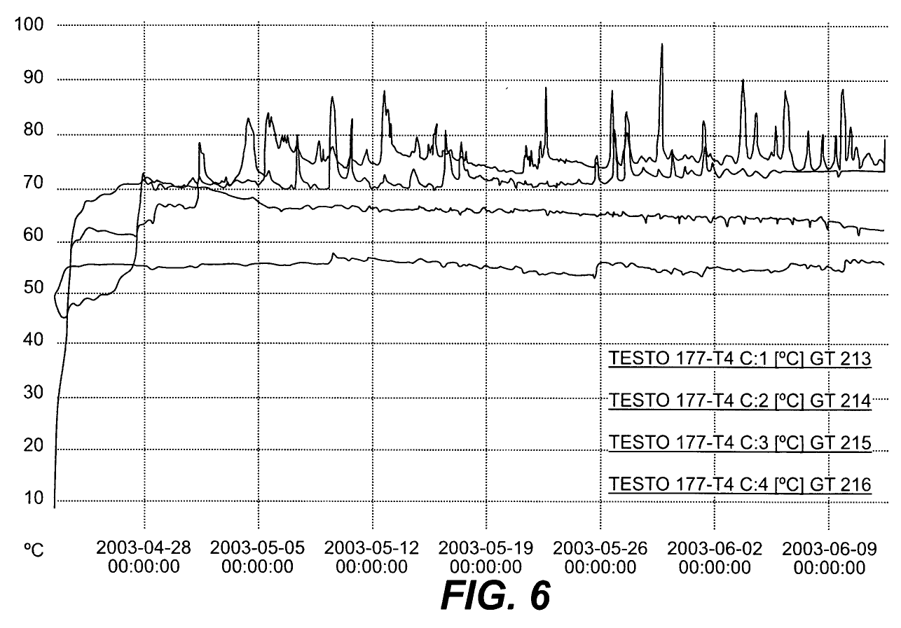

A covering layer can avoid the creation of an outer cooler edge zone in the matrix where a satisfactory sanitizing might not be achieved. A closed process thus can avoid the necessity in an open process to blend the material several times during the process to ensure a homogenously sanitized end product. During the closed process the turning of the material is not required to achieve suitable temperature development. See FIG. 6. A closed process has the additional advantage to minimize, among other things, the loss of gases from the microbial transformation, like methane and nitrogenous gases; for example, sulfur dioxide and ammonia, and thus it can also minimize the odor release into the surrounding environment or neighborhood.

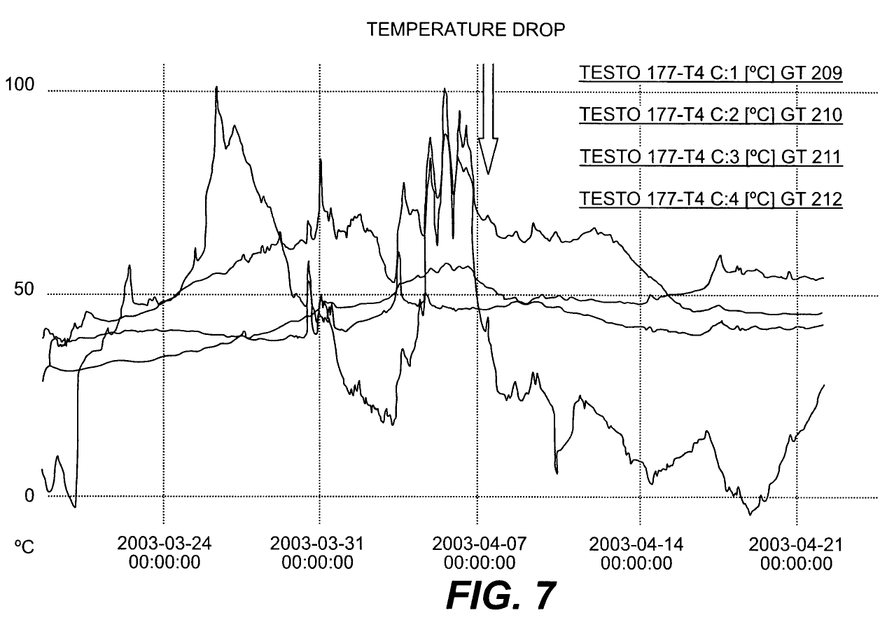

In certain embodiments, the present invention can involve the homogenization of the matrix. To avoid uneven temperature distribution, the matrix thus can be sufficiently mixed so that it achieves suitable amount of aeration and distribution within it at the mixing stage of the sanitization process. Appropriate homogenization can also prevent a risk of fire during mixing and aeration, in particular with regard to very nutrient rich material Such as, for example, knot reject, leading to a premature loss of carbon. See. FIG. 7. To enhance homogeneity, the outer material of the matrix in an open process can be well blended into the matrix. To ensure that the material is substantially sanitized, therefore, the material in a matrix in an open process can be blended several times during the sanitization. To achieve the proper sanitary level, the matrix in the open process can be turned around 3 times or more, for example, during the course of sanitization.

The appropriate homogenization to accomplish sanitizing the organic waste according to certain embodiments of the present invention can also take into account the prevailing climatic conditions in the area where the treatment is performed. Significant for cold climate conditions, can be that whereas the mixing leads to better contact between the organic waste and the nutrient rich organic material and facilitates aeration, which is positive for temperature development and the sanitizing process, it may lead to losses of heat.

The sanitized products produced by the present invention are hygienic, i.e., the products meet the appropriate sanitary level that they can be used in, for example, agriculture in such a way as to prevent or minimize harmful or unwanted effects on soil, vegetation, humans and other animals. The appropriate sanitary level can be, for example, products that do not contain viable Salmonella spp in 50 g (wet weight) or the products of treatment that has achieved at least a 6 Log₁₀ reduction in Escherichia coli to less than 5×10² CFU/g. See Annex I of the Working Document on Sludge, 3^{rd} draft, EC 2000, ENV.E.3/LM., relating to the EC Directive 86/278/EEC.

The sanitized products of the present invention can be free of, or contain a nominal quantity of, harmful or unwanted microorganisms, such as pathogens. These products can be used advantageously alone or in combination with soil or humus and other nutrient-enriched products. Examples of applications for these products can include use as a soil product, such as a soil substitute, soil additive, and fertilizer. The sanitized organic waste can be mixed as a component to form an end-product, air-dried, dehydrated, pelletized, or packed in a variety of other forms to facilitate lighter weight and less voluminous transport of the product. As a complementary measure, the sanitized organic waste of the present invention also can be treated using other composting methods.

Various examples of sanitized organic waste use can include mulch-enhancing material within agriculture and gardening; soil component to planting-soil, plant-soil, industrial-soil, or the like; dress-material for golf courses, soccer courses, or the like; fertilizer; erosion-protectant; growth-layer over surfaces where one wants to establish a plant-layer; plant material as growth-substrates for plants, trees, and the like; CEC-enhancing material to maximize the nutritional binding capacity in soils; and an isolating-layer.

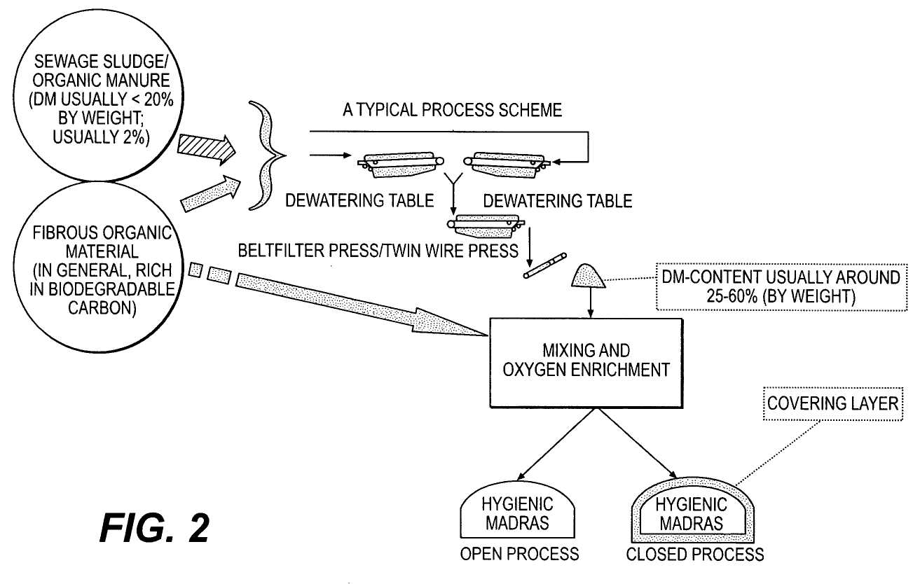

Untreated organic waste can contain a high percentage of water. Dewatering of sludge, for example, can be advantageous to achieve better structure, strength, and a higher degree of homogeneity. See FIG. 2. One embodiment of the present invention can be a method of dewatering organic waste without introducing exogenous chemicals, such as polymers, which are conventional additives in sludge dewatering, for example. Another embodiment of the present invention can be a method of dewatering organic waste by minimizing the amount of exogenous chemicals used to achieve sludge dewatering, for example. Yet another embodiment of the present invention can be a method of dewatering organic waste to promote insignificant odor and to achieve a high dry matter content. The organic fibrous material in the mixtures obtained by the dewatering methods of the present invention may also be further used as nutrient rich material facilitating the sanitizing process.

The methods of dewatering organic waste can be optimized by varying a mixture of organic waste and organic fibrous material. By using the dewatering method of the present invention, the amount of exogenous chemicals introduced in the dewatering methods of the present invention can be reduced by about 10% to 70% of that otherwise necessary to achieve acceptable dewatering. In an embodiment, for example, the present invention utilizes the application of at least 5% by weight of organic fibrous material, calculated on the dry matter content in the dewatered fraction. About 5-60% by weight of organic fibrous material such as, for example, cellulose, paper reject, dissolved or not dissolved papers and boards, bark, fiber sludge, park waste, and agricultural products or byproducts, or the like, can be added to organic waste and then dewatered. Belt filter press, screw press, centrifuge, twin wire press, filter press, rotary drum, vacuum filter, cementation tanks or pounds are various examples of dewatering equipment that may be used to dewater the organic material according to the present invention.

The dry matter content of the dewatered material, when the material is well blended and homogenous, can be about 20-50%. Attendant odors can be reduced. Where desired, dewatering can occur immediately preceding, or well in advance, of the sanitizing method of the present invention. As an example, before the organic waste (e.g., sewage sludge) is blended into the matrix, the organic waste can be dewatered at a sewage-treatment plant.

The following examples reflect certain embodiments of the present invention. Other embodiments of the invention will be apparent to those skilled in the art from a consideration of the specification and the practice of the invention disclosed herein, and thus, are within the spirit and scope of the invention.

### EXAMPLE 1

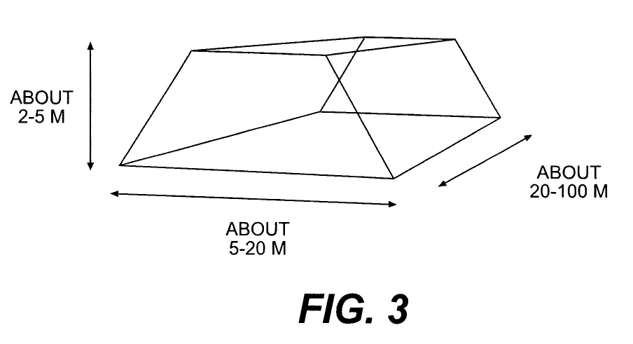

The organic waste sanitized was sewage sludge and the nutrient rich organic material used was bark. Sewage sludge was mixed with an equal amount of bark and, thus, the dry matter content of the mixture comprised 50% by volume of sewage sludge and 50% by volume of bark. The components were then formed into a porous matrix. The sewage sludge was thoroughly mixed with the bark using a digger, which ensured excellent oxygen enrichment to the matrix, the mixture was arranged into a sanitizing matrix of 10,000 m³ in size. FIG. 3.

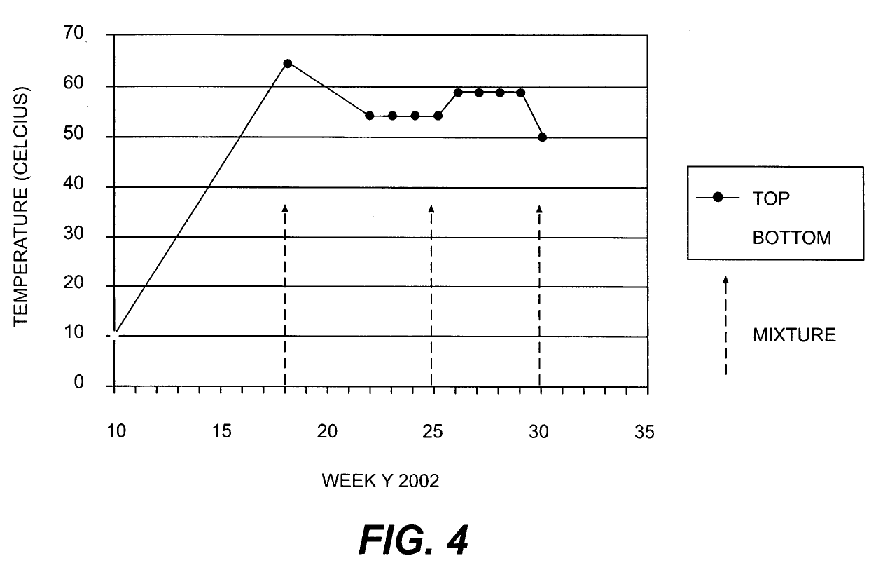

The matrix was maintained without introducing exogenous heat or chemicals, yet the temperature rose over time with the microbial transformation of the material. Although the cooler earlier spring weather of the northern hemisphere affected the experiment, at the peak of transformation during week 18 the temperature reached 65° C. FIG. 4. A temperature close to 60° C. was maintained at the top of the matrix during weeks 26-29.

The treated representative soil samples that were used for analysis did not contain any viable Salmonella spp in 50 g (wet weight). Compared to the initial samples taken from the matrix, the treatment attained a 6 Log₁₀ reduction in Escherichia coli and the Escherichia coli were present in less than 5×10² CFU/g.

TABLE 1

| Untreated organic waste | | Sanitized organic waste | |
|---|---|---|---|
| E. coli | 1900000/g | E. coli | <100/g |
| Streptococes | 150000/g | Streptococes | ˜100/g |
| Salmonella | Positive per 25 g | Salmonella | Negative per 25 g |

The E. coli measuring method was SS 028167/78; the Streptococes measuring method was SS 028179/78; the Salmonella measuring method was ISO 6340.

### EXAMPLE 2

The organic waste was sewage sludge and the organic fibrous material contained fiber sludge and bark. Sewage sludge was dewatered by adding organic fibrous material without significant addition of chemicals, such as polymers, due to the fibrous content of the slurry. About 15% by weight of bark calculated from the dewatered sewage sludge was added to the sewage sludge before centrifuging the mixture.

The dewatered sludge was then mixed with additional bark and fiber sludge so that the dry matter content of the mixture comprised about 33% by volume of sewage sludge, about 33% by volume of bark, and about 33% by volume of fiber sludge. The components were then formed into a porous matrix by thoroughly mixing the sewage sludge with the bark and the fiber sludge (ensuring a good oxygen enrichment of the matrix), and arranging the mixture into a sanitizing matrix of 1000 m³ in size.

The matrix was maintained without introducing exogenous heat or chemicals. The microbial transformation of the material raised the temperature to a peak of 70° C., which was maintained at the top of the matrix for the 24 hours to achieve the desired sanitary level. A sample of the sanitized organic waste was analyzed and did not contain any viable Salmonella spp in 50 g (wet weight) and showed a 6 Log₁₀ reduction in Escherichia coli (to less than 5×10² CFU/g) as compared to a sample taken from the matrix.

### EXAMPLE 3

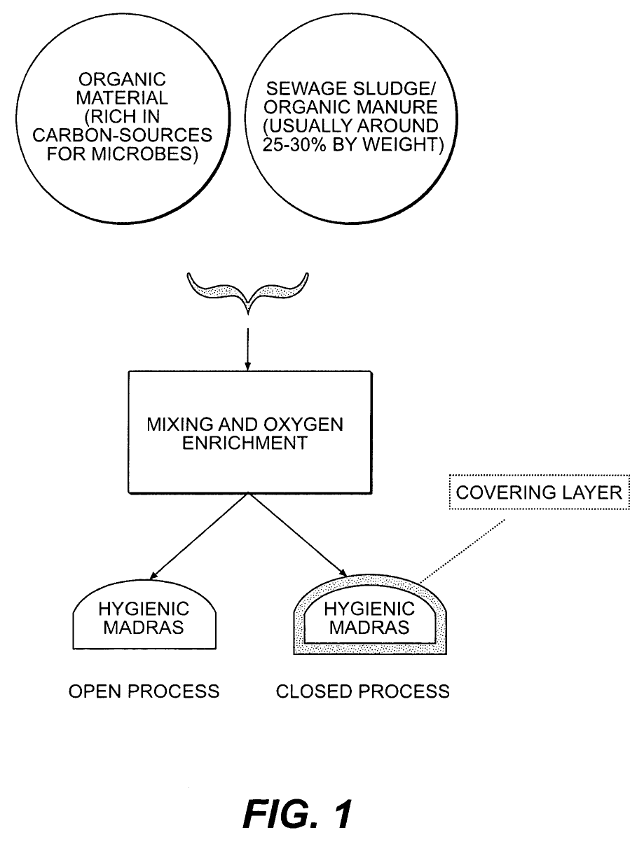

The organic waste was sewage sludge and the nutrient rich organic material contained fiber sludge and bark. The organic waste was treated using an open process and the mixture was homogenized prior to recovering the sanitized mixture. See FIG. 1. The sewage sludge was mixed with fiber sludge and bark to obtain a mixture that comprised about 33% by volume sewage sludge, about 33% by volume of fiber sludge, and about 33% by volume of bark. The components were then formed into a porous matrix by thoroughly mixing the sewage sludge with the fiber sludge (ensuring a good oxygen enrichment of the matrix), and arranging the mixture into a sanitizing matrix of 500 m³ in size.

The matrix was maintained without introducing exogenous heat or chemicals. The elevated temperature due to the microbial transformation of the material was reached higher up in the matrix and maintained at 65° C. for 72 hours. The material was then homogenized by turning around the mixture. The mixture was then turned around an additional two times and maintained at 65° C. for 72 hours. After the mixture was turned around for the third time, the mixture was maintained at 65° C. for 72 hours prior to recovering the sanitized mixture. A sample of the sanitized organic waste was analyzed and did not contain any viable Salmonella spp in 50 g (wet weight) and showed a 6 Log₁₀ reduction in Escherichia coli (to less than 5×10² CFU/g) as compared to a sample taken from the matrix.

### EXAMPLE 4

The organic waste was sewage sludge and the nutrient rich organic material contained fiber sludge and bark. The organic waste was treated using a closed process and the mixture was not homogenized prior to recovering the sanitized mixture. See FIG. 1. The sewage sludge was mixed with fiber sludge and bark to obtain a mixture that comprised about 33% by volume sewage sludge, about 33% by volume of fiber sludge, and about 33% by volume of bark. The components were then formed into a porous matrix by thoroughly mixing the sewage sludge with the fiber sludge (ensuring a good oxygen enrichment of the matrix), and arranging the mixture into a sanitizing matrix of 1000 m³ in size.

The matrix was then covered by a layer of bark, thereby shielding the mixture from, for example, rain. An advantageous effect of the covering layer was its insulation of the heat generated by the microbes in the mixture. The matrix was maintained without introducing exogenous heat or chemicals. With respect to this matrix, the elevated temperature that was attained higher up in the matrix was maintained at 60° C. for 6 days. Because the process was closed, there was no need to turn the mixture around. A sample of the sanitized organic waste was analyzed and did not contain any viable Salmonella spp in 50 g (wet weight) and showed a 6 Log₁₀ reduction in Escherichia coli (to less than 5×10² CFU/g) as compared to a sample taken from the matrix.

### EXAMPLE 5

To verify the sanitization effect of the methods of the present invention, several experiments were carried out that confirmed, for example, 100% sanitization effect to the lowest detectable level of the investigated pathogens. In addition, the strictest proposal regarding sanitization requirements in Sweden (class A) has been met.

TABLE 2

| Year and | Components - | | | | | |
|---|---|---|---|---|---|---|
| Trial nr. | Other then | | | Before the | After the | |
| (Starting | sewage | | Pathogens | hygienic | hygienic | |
| date) | sludge | Description | investigated | process* | process* | Reference |
| 2002-01 | +50 vol-% | Mixing 3 | E.. coli: | 1900000 | <100 | 2002-01570/ |
| (09-04) | bark | times/week | F. Streptokokker: | 150000 | <100 | 2002-01757 |
| | | | Salmonella sp.: | Proved | Not proven | |
| | | | DM (%): | 33.0 | 42.2 | |
| | | | Sample taken: | 020905 | 020927 | |
| 2002-02 | +50 vol-% | Mixing 3 | E.. coli: | 1900000 | <100 | 2002-01570/ |
| (09-09) | fiber sludge | times/week | F. Streptokokker: | 150000 | <100 | 2002-01758 |
| | | | Salmonella sp.: | Proved | Not proven | |
| | | | DM (%): | 33.0 | 32.2 | |
| | | | Sample taken: | 020905 | 020927 | |
| 2002-03 | +50 vol-% | Mixing 1 | E.. coli: | 1900000 | | 2002-01570 |
| (09-09) | bark | time/week | F. Streptokokker: | 150000 | | |
| | | | Salmonella sp.: | Proved | | |
| | | | DM (%): | 33.0 | | |
| | | | Sample taken: | 020905 | | |
| 2002-04 | +50 vol-% | Mixing 1 | E.. coli: | 1900000 | | 2002-01570 |
| (09-10) | fiber sludge | time/week | F. Streptokokker: | 150000 | | |
| | | | Salmonella sp.: | Proved | | |
| | | | DM (%): | 33.0 | | |
| | | | Sample taken: | 020905 | | |
| 2002-05 | +50 vol-% | Mixing 1 | E.. coli: | 1900000 | | 2002-01570 |
| (09-18) | fiber sludge | time/month | F. Streptokokker: | 150000 | | |
| | | | Salmonella sp.: | Proved | | |
| | | | DM (%): | 33.0 | | |
| | | | Sample taken: | 020905 | | |
| 2002-06 | +50 vol-% | Mixing 1 | E.. coli: | 1900000 | | 2002-01570 |
| (09-18) | bark | time/month | F. Streptokokker: | 150000 | | |
| | | | Salmonella sp.: | Proved | | |
| | | | DM (%): | 33.0 | | |
| | | | Sample taken: | 020905 | | |
| 2002-07 | +25 vol-% | Mixing 3 | E.. coli: | 1900000 | | 2002-01570 |
| (10-07) | bark | times/week | F. Streptokokker: | 150000 | | |
| | +25 vol-% | With 2400 kg | Salmonella sp.: | Proved | | |
| | fiber sludge | Mineral | DM (%): | 33.0 | | |
| | | fertilizer | Sample taken: | 020905 | | |
| 2002-08 | +25 vol-% | Mixing 3 | E.. coli: | 1900000 | | 2002-01570 |
| (10-07) | bark | times/week | F. Streptokokker: | 150000 | | |
| | +25 vol-% | Without | Salmonella sp.: | Proved | | |
| | fiber sludge | Mineral | DM (%): | 33.0 | | |
| | | fertilizer | Sample taken: | 020905 | | |
| 2002-09 | +50 vol-% | Mixing 1 | E.. coli: | 1900000 | <100 | 2002-01570/ |
| (10-14) | bark | time before | F. Streptokokker: | 150000 | <100 | 2003-00137 |
| | | entering the | Salmonella sp.: | Proved | <1 | |
| | | hygienic | DM (%): | 33.0 | 34.7 | |
| | | madras (closed | Sample taken: | 020905 | 030127 | |
| | | process- | | | | |
| | | fiber sludge) | | | | |
| 2002-10 | +50 vol-% | Material | E.. coli: | 1900000 | <1000 | 2002-01570/ |
| (10-14) | bark | from trial | F. Streptokokker: | 150000 | <100 | 2003-00138 |
| | | 2002-03 | Salmonella sp.: | Proved | <1 | |
| | | (closed | DM (%): | 33.0 | 41.9 | |
| | | process- | Sample taken: | 020905 | 030127 | |
| | | fabrics) | | | | |
| 2003-01 | +50 vol-% | 102.8 tonne | E.. coli: | 37-46 * 10⁶ | <100 | 2003-00222-2003-00223/ |
| (02-11) | bark | 150 m³ | F. Streptokokker: | 2-3.7 * 10⁶ | 100 | 2003-00696 |
| | | | Salmonella sp.: | Proven | Not proven | Testo id: |
| | | | DM (%): | 26.3-24.7 | 38 | 00743208 |
| | | | Sample taken: | 030213 | 030422 | |
| 2003-02 | +50 vol-% | 99.1 tonne | E.. coli: | 37-46 * 10⁶ | <100 | 2003-00222-2003-00223/ |
| (02-11) | fiber sludge | 150 m³ | F. Streptokokker: | 2-3.7 * 10⁶ | <100 | 2003-00697 |
| | | | Salmonella sp.: | Proven | Not proven | Testo id: |
| | | | DM (%): | 26.3-24.7 | 40.4 | 00743210 |
| | | | Sample taken: | 030213 | 030422 | |
| 2003-03 | +50 vol-% | 81.65 tonne | E.. coli: | 37-46 * 10⁶ | <100 | 2003-00222-2003-00223/ |
| (02-12) | knot reject | 150 m³ | F. Streptokokker: | 2-3.7 * 10⁶ | 100 | 2003-00698 |
| | | | Salmonella sp.: | Proven | Not proven | Testo id: |
| | | | DM (%): | 26.3-24.7 | 39.8 | 00743216 |
| | | | Sample taken: | 030213 | 030422 | |
| 2003-04 | +50 vol-% | 92.65 tonne | E.. coli: | 37-46 * 10⁶ | <100 | 2003-00222-2003-00223/ |
| (02-12) | park debris | 150 m³ | F. Streptokokker: | 2-3.7 * 10⁶ | <100 | 2003-00699 |
| | | | Salmonella sp.: | Proven | Not proven | Testo id: |
| | | | DM (%): | 26.3-24.7 | 49.5 | 00743227 |
| | | | Sample taken: | 030213 | 030422 | |
| 2003-05 | +17 vol-% | 96.45 tonne | E.. coli: | 37-46 * 10⁶ | <100 | 2003-00222-2003-00223/ |
| (02-12) | bark + 17 | 150 m³ | F. Streptokokker: | 2-3.7 * 10⁶ | <100 | 2003-00819 |
| | vol-% fiber | | Salmonella sp.: | Proven | Not proven | Testo id: |
| | sludge + 17 | | DM (%): | 26.3-24.7 | 39.2 | 00715365 |
| | vol-% | | Sample taken: | 030213 | 0305122 | |
| | knot reject | | | | | |
| 2003-06 | +25 vol-% | 103.9 tonne | E.. coli: | 37-46 * 10⁶ | <100 | 2003-00222-2003-00223/ |
| (02-12) | bark + 25 | 150 m³ | F. Streptokokker: | 2-3.7 * 10⁶ | <100 | 2003-00820 |
| | vol-% park | | Salmonella sp.: | Proven | Not proven | Testo id: |
| | debris | | DM (%): | 26.3-24.7 | 39.5 | 00743172 |
| | | | Sample taken: | 030213 | 030512 | |
| 2003-07 | +25 vol-% | 100.75 | E.. coli: | 37-46 * 10⁶ | | 2003-00222-2003-00223/ |
| (02-12) | bark | tonne | F. Streptokokker: | 2-3.7 * 10⁶ | | |
| | | 150 m³ | Salmonella sp.: | Proven | | |
| | | | DM (%): | 26.3-24.7 | | |
| | | | Sample taken: | 030213 | | |
| 2003-08 | +75 vol-% | 94.8 tonne | E.. coli: | 37-46 * 10⁶ | <100 | 2003-00222-2003-00223/ |
| (02-12) | bark | 150 m³ | F. Streptokokker: | 2-3.7 * 10⁶ | <100 | 2003-00821 |
| | | | Salmonella sp.: | Proven | Not proven | Testo id: |
| | | | DM (%): | 26.3-24.7 | 35.8 | 00743179 |
| | | | Sample taken: | 030213 | 030512 | |
| 2003-09 | +67 vol-% | App. 175 m³ | E.. coli: | 37-46 * 10⁶ | <100 | 2003-00222-2003-00223/ |
| (04-22) | knot reject | With or | F. Streptococci: | 2-3.7 * 10⁶ | <100 | 2003-01285-2003-01284 |
| | (41.6% DM) | without | Salmonella sp.: | Proven | Not proven | Testo id: |
| | | GORE-e.g. | DM (%): | 26.3-24.7 | 44.2-49.9 | 00743227 |
| | | Fabrics | Sample taken: | 030213 | 030715 | 00743216 |
| 2003-10 | +67 vol-% | App. 175 m³ | E.. coli: | 37-46 * 10⁶ | <100 | 2003-00222-2003-00223/ |
| (04-22) | knot reject | With or | F. Streptokokker: | 2-3.7 * 10⁶ | <100 | 2003-01287-2003-01286 |
| | (54.2% DM) | without | Salmonella sp.: | Proven | Not proven | Testo id: |
| | | GORE- e.g. | DM (%): | 26.3-24.7 | 45.4-43.1 | 00743210 |
| | | fabrics | Sample taken: | 030213 | 030715 | 00743208 |
| 2003-11 | +67 vol-% | App. 100 m³ | E.. coli: | 37-46 * 10⁶ | <100 | 2003-00222-2003-00223/ |
| (05-05) | knot reject | With sealed | F. Streptokokker: | 2-3.7 * 10⁶ | <100 | 2003-01288 |
| | (54.2% DM) | tarpaulin | Salmonella sp.: | Proven | Not proven | 2003-01289 |
| | | | DM (%): | 26.3-24.7 | 45.4-43.1 | Testo id: |
| | | | Sample taken: | 030213 | 030715 | 00743172 |
| 2003-12 | +33 vol-% | App. 2 * 150 m³ | E.. coli: | 100 | | 2003-00822 |
| (06-02) | knot reject | Comparison | F. Streptococci: | 430 | | Testo id: |
| | +33 vol-% | with/without | Salmonella sp.: | Proven | | 00743208 |
| | bark | mixing | DM (%): | 22.3 | | 00743210 |
| | | equipment | Sample taken: | 030512 | | 00743216 |
| | | on the | | | | 00743227 |
| | | bucket | | | | |
| 2003-13 | A. 67 vol-% | A. 500 m³ | E.. coli: | Not to be | | Testo id: |
| (06-23) | bark | B. 500 m³ | F. Streptococci: | taken | | 00715365 |
| | B. 500 m³ | Investigate | Salmonella sp.: | | | 00743172 |
| | 67 vol-% | temperature | DM (%): | | | 00743179 |
| | fiber sludge | distribution | Sample taken: | | | 00778170 |
| | | and optimal | | | | |
| | | size | | | | |

“*” Refers to the number of coliform units per gram DM.
“DM” refers to the dry-matter content. “Testo id” refers to the temperature gauges id number.

### EXAMPLE 6

Experiments were carried out on two different mixtures of organic waste and nutrient rich organic material. Each mixture comprised the same kind of organic waste and nutrient rich organic material, which was blended to confirm optimal mixing intervals to achieve appropriate aeration for the sanitization process. Three experiments were carried out regarding each mixture-blend. The first mixture comprised the organic waste material sewage sludge (50 vol-%) and the nutrient rich organic material bark (50 vol-%). The second mixture comprised the organic waste sewage sludge (50 vol-%) and the nutrient rich organic material fiber sludge (50 vol-%). The materials were mixed three times per week, once weekly, and once monthly, respectively.

During the fall season's climate condition and the mixing technique used (bucket on a loader), the optimal interval was frequently found to be once weekly and based on a period of 50-64 days. The difference with respect to the sanitization effect between the three times a week and the once weekly mixings was insignificant, in particular compared to mixing once monthly.

The experiments carried out at colder conditions (during winter season) indicated that a less frequent mixing was preferable compared to the mixing interval of once weekly. During such weather condition and using such blending techniques, the optimal mixing interval should likely be 1-2 times per month.

Accordingly, the optimal interval for mixing and aeration likely varies from once weekly (during warmer climate conditions) to 1-2 times per month (during colder climate conditions

TABLE 3

| a. Material | Depth | 3 times/w | 1 time/w | 1 times/m |
|---|---|---|---|---|
| 50 vol-% sewage sludge + | 0.5 m | 62 | 62 | 54 |
| 50 vol-% bark | 1.5 m | 60 | 60 | 54 |
| 50 vol-% sewage sludge + | 0.5 m | 50 | 51 | 48 |
| 50 vol-% fiber sludge | 1.5 m | 45 | 47 | 43 |

The average temperature in degrees Celsius during mixing and aeration, fall season, at Valsta, Sweden.

### EXAMPLE 7

The main focus was to compare the efficiency of nutrient rich organic materials to generate heat to accomplish sanitizing the organic waste. Some materials worked better and quickly generated high temperatures within the matrix, whereas other materials, although suitable, were less efficient.

Nutrient rich organic material having a high content of, or being presumed to be high in content of, biodegradable carbon was used. To evaluate the efficiency of the materials, VOS based laboratory tests were carried out regarding six different materials. “VOS” refers to “Våmvätskelöslig Organisk Substans”, which demonstrates the amount of organic content that is biodegradable in liquid from ruminants (sheep) within 96 hours. The results from the laboratory tests generally confirmed that, for example, knot reject is the preferable material to achieve high temperature and thereby sufficient sanitization.

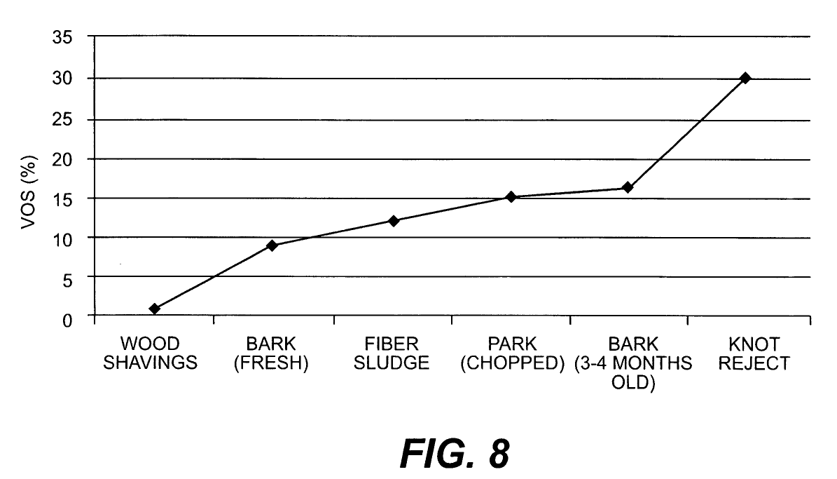

Several of the nutrient rich organic materials performed very well, for example, knot reject, bark, and various by-products from garden restoration. Fiber sludge was also sufficient and less efficient, although potentially usable, were, for example, wood chips and the like materials. In order of efficiency, it was found that knot reject is most efficient, thereafter bark (not too old) and park debris (preferably chopped), and finally fiber sludge. See FIG. 8.

### EXAMPLE 8

The nutrient rich organic materials proved efficient for heat generation were tested and demonstrated relatively minor differences with respect to the temperature development within the matrix. The temperature generally, but not always, seemed to follow a ratio of the added volume percentage of the nutrient rich organic material to the sewage sludge in degrees Celsius.

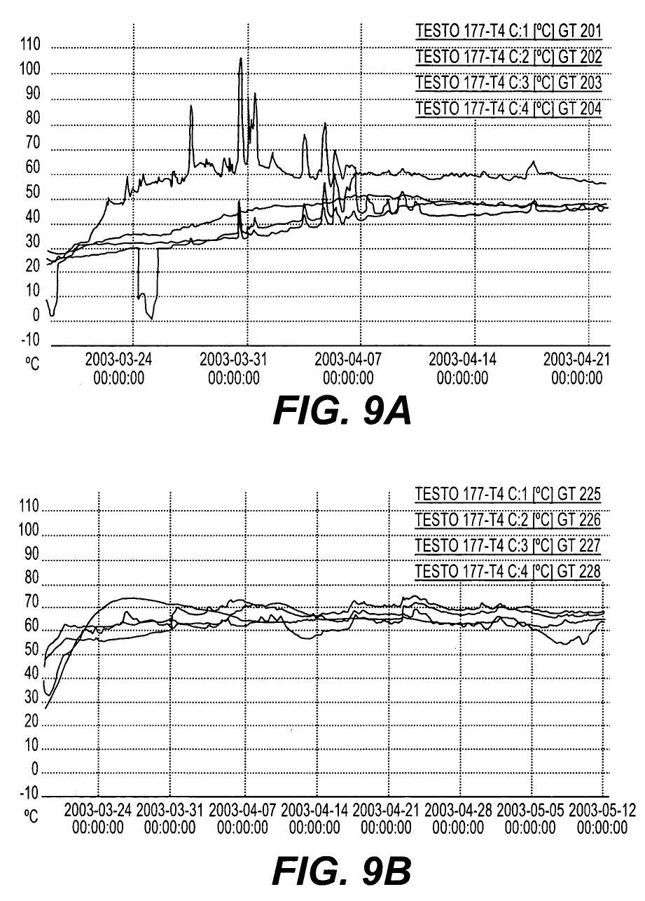

The above approximate formula would, for example, estimate that an added volume of 50% bark presumably results in a temperature of about 50 degrees Celsius within the matrix. Because an added volume of nutrient rich organic material to the mixture approximately increased the temperature accordingly, a volume of, for example, 60-70% nutrient rich organic material should likely result in a temperature of around 60-70 degrees Celsius, etc. See FIG. 9.

### EXAMPLE 9

Different covers were tested with respect to isolation, protection, and environmental considerations. The organic materials fiber sludge and knot reject as well as the inorganic material GORETEX-sheet were tested. The optimal thickness of the cover seemed to be about 0.5 meters.

A thick cover could necessitate a large amount of material that, for example, risked an increase of weight upon the matrix. Because an increased weight upon the matrix can reduce the amount of oxygen and also affect its porosity, an excessively thick and heavy cover negatively can interfere with the sanitization process by reducing the temperature. The prevailing climatic conditions, such as winter season, may require a sufficiently thick cover to decrease the risk of a colder outer layer. Generally, a thinner cover meant a greater loss of temperature from the matrix.

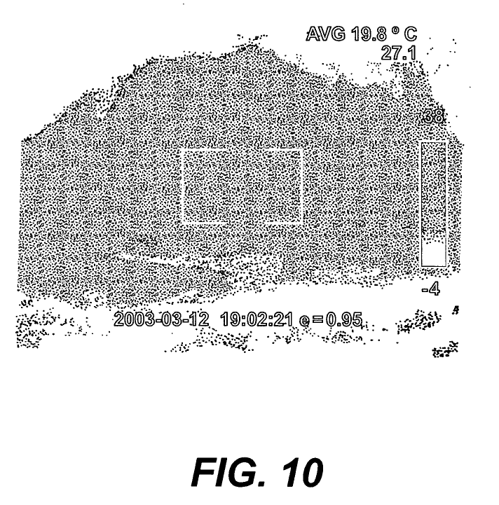

The temperature analyses indicated that the optimal thickness for a cover consisting of fiber sludge and knot reject was about 0.5 meters. The temperature peak that was frequently found 0.5 meters from the top surface in several matrixes confirms this finding. The temperature at the outermost layer and in the central area of the matrix was critical. A typical temperature distribution in a matrix demonstrating the two factors is clear from FIG. 10. The potential difficulty to achieve the desired temperature in the outermost layer even in colder weather conditions was not found using a closed process.

### EXAMPLE 10

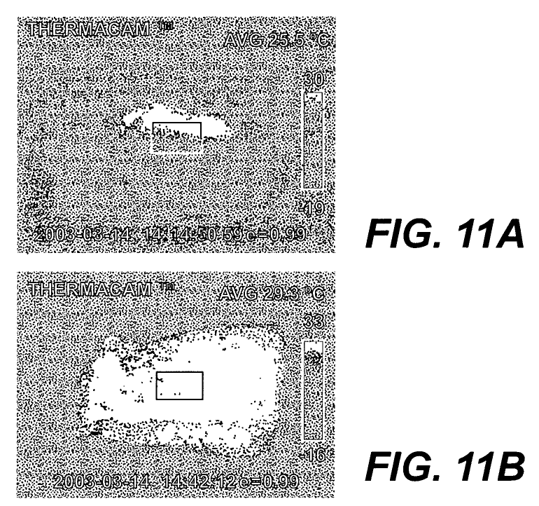

The achievement of desired temperature in the central area of the matrix was tested. The temperature variation within the matrix was greater in the beginning of the sanitization process compared to later on and in its final stage. This was likely due to a higher content of water and heavier material at the early stage of the process that compressed oxygen from the central of the matrix, therefore aggravating the process. See FIG. 11.

### EXAMPLE 11

The effect of applying mineral fertilizer to the matrix was tested. In the tested proportion between sewage sludge and nutrient rich organic material this addition to the matrix of the fertilizer had no or very little effect with respect to temperature development during the sanitization process. Either with and without adding extra mineral fertilizer to the mixture in the beginning of the sanitization process was tested. The amount of mineral fertilizers was in the order of 2.400 kg of NPK (i.e., nitrogen, phosphors, and potassium) including micronutrients to the matrixes, which approximately weighted 120 tones, consisting of 150 m³ of fresh raw material. The added mineral fertilizer had no or insignificant effect on the temperature development.

What is claimed is:

1. A method for sanitizing organic waste, comprising:
mixing organic waste with nutrient rich organic material to thereby form a mixture;
forming a porous matrix comprising the mixture of organic waste and nutrient rich organic material;
maintaining the matrix, without introducing exogenous heat or chemicals, at a temperature and duration sufficient to reduce the amount of living microorganisms in the mixture to sanitary levels; and
recovering a sanitized mixture.

2. The method according to claim 1, wherein the organic waste is selected from the group consisting of organic manure, contaminated soil, residual sludge from sewage plants treating domestic or urban wastewater as well as from other sewage plants treating wastewater of a composition similar to domestic and urban wastewater, residual sludge from septic tanks and other similar installations for the treatment of sewage, residual sludge from other sewage plants or sources sludge derived from industrial products and byproducts comprising organic material, including sludge from food-processing industry, sludge from otherwise biodegradable materials, not necessarily of biological origin, an organic sludge composed of materials such as sludges resulting from production of antimicrobials and other pharmaceutical products, bacterial fermentation sludges, sludges resulting from production of beer and wine, mushroom compost waste, paper mill sludges, sludges that contain microorganisms resulting from recycled organic products such as paper products, sludges resulting from the growth of microorganisms for the production of chemicals and organics, industrial sludges and byproducts resulting from the production of microbial products and foodstuffs, sludges resulting from the animal slaughter industry, includes those that are digested or otherwise broken down by microorganisms, and sludges containing animal manure, including at least one of chicken, pig, cattle, and horse manure, and mixtures of the above sludges and materials.

3. The method according to claim 1, wherein the organic waste is selected from the group consisting of sludge, soil contaminated by oil, soil contaminated by grocery-industry products or byproducts, contaminated animal-feed, cadavers and butcher waste, cattle feedlot and swine manure, and composted manure used in organic farming practices.

4. The method according to claim 1, wherein the mixture comprises about 30% to 80% nutrient rich organic material.

5. The method according to claim 1, wherein the nutrient rich organic material is selected from the group consisting of plant residues, products from forest and paper industry including tree bark, debarking reject, knot reject, fiber sludge, bio sludge, chemical sludge, mixed sludge, wood working shop reject and factory second rejected products, screen reject, recycled fiber including dip sludge, screenings, and wood chops, products from saw-mill industry including saw dust and planning shavings, products from agriculture including hay, straw, and other crop residues from grain, sugar cane, maize, and rice, products from other food-processing industries including whey, peel, coco fiber, and products from vegetables and fruit processing industry including rice reject and olive seeds, garden and tree waste, and peat.

6. The method according to claim 1, further comprising adding to the mixture an adjusting component selected from the group consisting of chalk, limestone, lime sludge, lime mud, burnt lime mud, green liquor sludge, ashes, mineral fertilizer, and material from fresh or sea water that is rich in nutrients, or solutions, suspension or other mixtures thereof.

7. The method according to claim 6, wherein the material from the fresh or sea water is selected from the group consisting of algae and organic manure.

8. The method according to claim 1, further comprising the step of covering the matrix, wherein said covering step precedes the step of maintaining the matrix, without introducing exogenous heat or chemicals, at a temperature and duration sufficient to reduce the amount of living microorganisms in the mixture to sanitary levels.

9. The method according to claim 1, further comprising the step of homogenizing the mixture, wherein said homogenizing step precedes the step of recovering a sanitized mixture.

10. The method according to claim 1, wherein the organic waste is sewage sludge and the nutrient rich organic material is bark, wherein the bark comprises 65% of the mixture and the sewage sludge comprises 35% of the mixture.

11. A sanitized mixture obtained by the method of claim 1.

12. A method of dewatering organic waste, comprising:
mixing organic waste with organic fibrous material sufficient to promote insignificant odor and high dry matter content, or without introducing significant amounts of exogenous chemicals; and
dewatering said mixture.

13. The method according to claim 12, wherein the organic waste is selected from the group consisting of organic manure, contaminated soil, residual sludge from sewage plants treating domestic or urban wastewater as well as from other sewage plants treating wastewater of a composition similar to domestic and urban wastewater, residual sludge from septic tanks and other similar installations for the treatment of sewage, residual sludge from other sewage plants or sources sludge derived from industrial products and byproducts comprising organic material, including sludge from food-processing industry, sludge from otherwise biodegradable materials, not necessarily of biological origin, an organic sludge composed of materials such as sludges resulting from production of antimicrobials and other pharmaceutical products, bacterial fermentation sludges, sludges resulting from production of beer and wine, mushroom compost waste, paper mill sludges, sludges that contain microorganisms resulting from recycled organic products such as paper products, sludges resulting from the growth of microorganisms for the production of chemicals and organics, industrial sludges and byproducts resulting from the production of microbial products and foodstuffs, sludges resulting from the animal slaughter industry, includes those that are digested or otherwise broken down by microorganisms, and sludges containing animal manure, including at least one of chicken, pig, cattle, and horse manure, and mixtures of the above sludges and materials.

14. The method according to claim 12, wherein the organic waste is selected from the group consisting of sludge, soil contaminated by oil, soil contaminated by grocery-industry products or byproducts, contaminated animal-feed, cadavers and butcher waste, cattle feedlot and swine manure, slurry from paper industry, and composted manure used in organic farming practices.

15. The method according to claim 12, wherein the mixture comprises about 5% to 60% by weight fibrous organic material.

16. The method according to claim 12, wherein the organic fibrous material is selected from the group consisting of cellulose, paper reject, dissolved or not dissolved papers and boards, bark, fiber sludge, park waste, and agricultural products or byproducts.

17. The method according to claim 12, further comprising adding to the mixture an adjusting component selected from the group consisting of chalk, limestone, lime sludge, lime mud, burnt lime mud, green liquor sludge, ashes, and material from fresh or sea water that is fibrous, or solutions, suspension or other mixtures thereof.

18. The method according to claim 1, further comprising the step of dewatering said organic waste prior to said mixing with nutrient rich organic material.

19. A method for sanitizing organic waste, comprising the steps of:
mixing organic waste with nutrient rich organic material to thereby form a mixture;
forming a porous matrix comprising the mixture of organic waste and nutrient rich organic material;
maintaining the matrix, without introducing exogenous heat or chemicals, at a temperature and duration sufficient to reduce the amount of living microorganisms in the mixture to sanitary levels, thereby generating a sanitized mixture; and
recovering the sanitized mixture.

20. The method according to claim 19, wherein the maintaining step includes periodically mixing the matrix to thereby aerate the organic waste and nutrient rich material.

21. The method according to claim 19, wherein the maintaining step includes covering the matrix with a cover.

22. The method according to claim 19, wherein the maintaining step includes maintaining the temperature of the matrix at a range of 50 to 90 degrees centigrade.

23. The method according to claim 19, wherein the organic waste is sludge or manure, and the nutrient rich material is selected from the group consisting of bark, knot waste, park debris, and fiber sludge.

24. The method according to claim 19, wherein the nutrient rich material comprises 60-70% of the matrix.

25. The method according to claim 20, wherein the periodically mixing step includes mixing the matrix between 1 and 4 times per month.

26. The method according to claim 19, wherein the sludge is dewatered sludge from a waste water treatment plant.

27. The method according to claim 19, further comprising the step of dewatering said organic waste prior to said mixing with nutrient rich organic material.

28. The method according to claim 19, further comprising the step of introducing sufficient amount of exogenous oxygen to the matrix to facilitate the sanitizing process.

29. A method for producing a sanitized mixture, comprising:
mixing organic waste with nutrient rich organic material to thereby form a mixture;
forming a porous matrix comprising the mixture of organic waste and nutrient rich organic material;
maintaining the matrix, without introducing exogenous heat or chemicals, at a temperature and duration sufficient to reduce the amount of living microorganisms in the mixture to sanitary levels;
recovering the sanitized mixture; and
dewatering said organic waste prior to said mixing with nutrient rich organic material.
